(19) United States
(12) Patent Application Publication
(10) Pub. No.: US 2019/0381215 A1
(43) Pub. Date: Dec. 19, 2019

(54) METHOD FOR SURFACE TREATMENT OF A DENTAL IMPLANT OR PROSTHETIC COMPONENT AND A DENTAL IMPLANT OR PROSTHETIC COMPONENT WITH A NANOPOROUS SURFACE

(71) Applicant: BIOTECHNOLOGY INSTITUTE, I MAS D, S.L., Vitoria (Alava) (ES)

(72) Inventor: Eduardo ANITUA ALDECOA, Vitoria (Alava) (ES)

(21) Appl. No.: 16/430,600

(22) Filed: Jun. 4, 2019

(30) Foreign Application Priority Data
Jun. 19, 2018 (ES) P 201830597

(51) Int. Cl. A61L 27/56; A61C 8/00; A61L 27/06; C25F 3/08; C25D 11/26

(52) U.S. Cl. CPC A61L 27/56; A61C 8/0015; C25D 11/26; C25F 3/08; A61L 27/06

(57) ABSTRACT

Method for the surface treatment of a dental implant or a prosthetic component made out of titanium or a titanium alloy, which enables an outer surface of the implant or the prosthetic component to be obtained with a notable capacity to prevent bacterial adhesion and offer a better aesthetic finish. This method comprises the steps of providing an outer surface of the implant or the prosthetic component with a surface roughness, and applying an anodizing treatment on the implant or the prosthetic component, smoothing the roughness and generating nanopores on this outer surface of the implant or the prosthetic component. The invention also relates to a dental implant or a prosthetic component made out of titanium or a titanium alloy, which comprises an outer surface that is rough and has nanopores.

### FIELD OF THE INVENTION

The invention relates to a method for surface treatment of a dental implant or prosthetic component, and in particular to a method that comprises the formation of a surface roughness on the dental implant or the prosthetic component and the subsequent anodisation of the dental implant or the prosthetic component to smooth this roughness, form nanopores on this roughness and provide the surface with a particular colouring. The invention also relates to a dental implant or prosthetic component with a rough outer surface with nanopores.

### PRIOR ART

Dental implants, generally made out of titanium, enable one or more dental prostheses to be anchored in partially or fully edentulous maxillary or jaw bones. This is possible thanks to the capacity of titanium to become osseointegrated, or in other words, to establish a direct and intimate interaction with the bone. Furthermore, titanium spontaneously forms an oxide surface layer that prevents the corrosion of the implant and its mechanical degradation when receiving forces as a result of its function.

In the field of dental implantology, the surface treatment of dental implants is known in the prior art in order to provide the implant surface with better properties that favour the integration of the implant in the bone tissue and therefore increase the success rates of the implantation. However, in spite of the development of the technique, there are still failures in the implantation of the implant for different causes.

It is known in the prior art that the fixation of dental implants is a complex process as there are three types of tissue involved: the epithelial tissue, the soft connective tissue and the bone. It has been described in literature that there are four foreseeable failure modes in transepithelial devices. The first consists of the recession of the soft tissue around the implant, creating a sac or vacuum. Secondly, the still immature connective tissue penetrates the pores of the implant, generating a lifting force in a process called “permigration”. This destabilising of the soft tissue breaks the protective seal around the implant and leaves a clear path for the potential entry of pathogens in this area between the implant and the soft tissue. The other two failure modes are infection and traumatic processes. Therefore, there is a clear consensus regarding the need to generate as tight a seal as possible in the interface between the soft tissue and the implant as an initial barrier against infection and as a vital factor for the long term success of implants and transepithelial components. In fact, the most common cause for failure in the implantation of dental implants lies in microbial colonisation in the area between the implant and the prosthesis. This cause of failure stands out from the rest as it is more common and may have important clinical implications.

For this reason, there is growing concern to maintain the soft gingival tissues and achieve a tight biological seal between the soft tissues and the surface of the implant and the prosthesis as this is crucial for the short and medium term success of the implantation. Gingival fibroblasts are the main ingredient of the periodontal tissue, and are responsible for maintaining the structural integrity of the connective tissues as well as providing a tight close of the soft tissue in the transmucosal part of the implant.

The accumulation of bacteria in this area between the implant, the dental prosthesis and the soft gingival tissues may cause the formation of biofilms, or in other words, an orderly accumulation of bacteria in thick layers in many individuals, which are resistant to treatment with antibiotics, and produce inflammatory diseases, such as peri-implant mucositis and peri-implantitis. Peri-implantitis is characterised by the loss of the supporting bone around the implant. It is estimated that peri-implantitis occurs in 6.6 to 36.6% of implants placed in the bone.

If the accumulation of bacteria is not prevented, any infections that occur as a result of this may require the removal of the implant and the affected tissues, and the subsequent cleaning and healing of the area before being able to insert a new implant. These operations involve additional costs and discomfort for the patient and may lead to serious health problems.

Therefore, it is essential to develop implant surfaces in the transmucosal and transepithelial area of the implant that reduce the initial number of adhered bacteria and hence, minimise the risk of the formation of plaque and the subsequent inflammation of the soft tissues.

In order to try to reduce the development of bacterial plaque in implants, numerous materials with different characteristics and surface treatments have been tested in the oral cavity. Some of these treatments contain metal ions, such as Ag⁺, Cu^{2 +}, Ni²⁺, Cr³⁺, Zn²⁺, Fe³⁺, etc., which have a bacterial effect, once released to the area around the implant. However, in components for prolonged implantation, this type of surfaces may cause a problem due to the accumulation of these metals in the blood. In these cases, the bacterial action of these metal ions is generally limited to the initial moments of the implantation and seeks the asepsis for surgery. For these metal ions to act over a longer period of time, there are ion “trapping” strategies in oxide layers so that they are only released when these protective layers are degraded. This occurs in those implants or prosthesis subjected to tribological phenomena, such as knee or hip prostheses.

On the other hand, there are treatments whose anti-bacterial effects are based on the surface texture. It is known that an increasing micro-roughness facilitates the formation of bacterial biofilms on the surfaces of implants and prosthetic components. On the other hand, modifications in the nano scale, through the inclusion of nanotubes or nanopores have proven to be very effective in the inhibition of bacterial adhesion. The techniques of obtaining these nanostructures, particularly those that produce more orderly structures, make their transfer to complex geometries difficult, such as the ordinary production of implants. Furthermore, these treatments usually have a greyish surface finish that is not very aesthetic for the desired use.

In order to give the transepithelial components favourable aesthetics for the subsequent prosthetic reconstruction, hard coatings have been made, such as those generated by titanium nitride plasma vapour deposition (PVD). However, its anti-bacterial effectiveness is limited and is due, mainly, to the low level of roughness and the fact that the surface hardening limits the release of ions.

It is therefore necessary to have anti-bacterial surfaces that fulfil these three requirements at the same time: that their bacterial activity is not based on the release of metal ions to the body which may accumulate in the organism; that they are aesthetically adapted to the prosthetic reconstruction; and they are inhibitors of the initial bacterial adhesion for the prevention of the formation of microbial biofilms and bacterial plaque that may jeopardise the implantation.

### BRIEF DESCRIPTION OF THE INVENTION

An object of the invention is a surface treatment method for a dental implant or prosthetic component made out of titanium or a titanium alloy, which enables an outer surface of the implant or prosthetic component to be obtained with a notable capacity to prevent bacterial adhesion. This method comprises the steps of providing an outer surface of the implant or prosthetic component with a surface roughness, and applying an anodising treatment on the implant or prosthetic component, smoothing the roughness and generating nanopores on this outer surface of the implant or prosthetic component. In other words, this invention proposes the combined use of smooth modification conditions of the surface texture with nanopores through the anodisation of surfaces previously made rough using other methods.

Another object of the invention is a dental implant or prosthetic component made out of titanium or a titanium alloy, which comprises a rough outer surface with nanopores.

The invention provides a dental implant or prosthetic component with greater resistance to bacterial adherence. Furthermore, the invention improves the aesthetic characteristics of the implantation of the implant or prosthetic component as the surface tones obtained allow for a better aesthetic finish of the implant or prosthetic component. Furthermore, the implant or prosthetic component according to the invention presents the advantage of not releasing metal ions. On the other hand, the implant and prosthetic component in this invention allows for a better fixing of the soft tissue (fibroblast adhesion). Finally, the method according to the invention avoids the use of fluorine compounds, which are the basis of obtaining nanotubes/nanopores in titanium in accordance with conventional methods and which present a high level of toxicity/risk in their handling.

### BRIEF DESCRIPTION OF THE FIGURES

The details of the invention can be seen in the accompanying figures, which do not intend to limit the scope of the invention:

FIG. 1 shows the visual appearance in accordance with the anodising voltage applied depending on the pre-existing surface: following the machining process, following the nitride additive treatment and following the subtractive acid treatment.

FIG. 2 shows the pore diameter distribution histogram in nm in depending on a selection of anodising voltages on samples with nitride additive treatment [A) 75V, B) 100V, C) 125V, D) 140V, E) 170V].

FIG. 3 shows the pore diameter distribution histogram in nm depending on a selection of anodising voltages on samples with subtractive acid treatment [A) 75V, B) 100V, C) 125V].

FIG. 4 shows scanning electron microscopy images which provide the topographic appearance in the micro and nano scale of the surfaces before and after the application of the same nano-texturised treatment [A) Surface after machining B) Surface after machining and anodising 100V C) Surface after subtractive acid treatment D) Surface after subtractive acid treatment and anodising 100V E) Surface after nitride additive treatment F) Surface after nitride additive treatment and anodising 100V].

FIG. 5 shows the results of the bacterial adhesion experiments with the Streptococcus Sanguinis (SS) and Staphylococcus Aureus (SA) strains in static conditions [A) Surface after machining B) Surface after machining and anodising 100V C) Surface after subtractive acid treatment D) Surface after subtractive acid treatment and anodising 100V E) Surface after nitride additive treatment F) Surface after nitride additive treatment and anodising 100V].

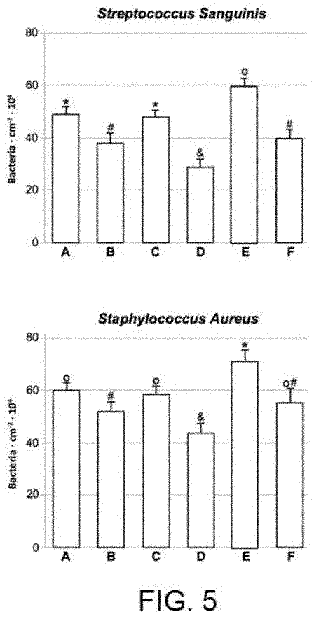

FIG. 6 shows the results of the bacterial adhesion experiments with the Streptococcus Sanguinis (SS), Streptococcus Mutans (SM) and Aggregatibacter Actinomycetemcomitans (AA) strains in dynamic conditions and conditioning in artificial or natural saliva [A) Surface after machining B) Surface after machining and anodising 100V C) Surface after subtractive acid treatment D) Surface after subtractive acid treatment and anodising 100V].

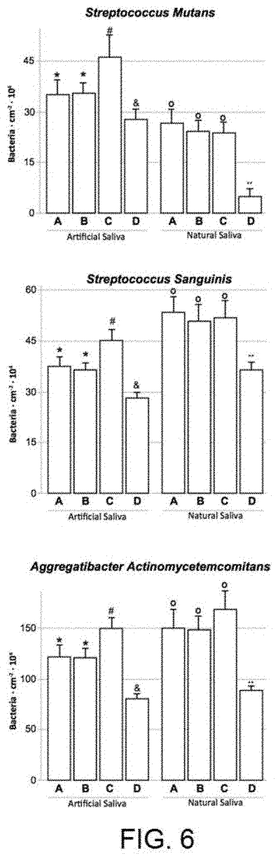

FIG. 7 shows the results of the DNA extraction experiments using metagenomics techniques, performed after 24 h of in vivo bacterial adhesion, displaying in the graph the results of the 6 most abundant bacteria found on the different surfaces [A) Surface after nitride additive treatment B) Surface after nitride additive treatment and anodising 100V].

FIG. 8 shows the results of the DNA extraction experiments using metagenomics techniques, performed after 24 h of in vivo bacterial adhesion, displaying in the graph the results of the 25 most pathogenic bacteria in relation to peri-implantitis phenomena found on the different surfaces [A) Surface after nitride additive treatment B) Surface after nitride additive treatment and anodising 100V].

FIG. 9 shows the results of the gingival-based primary fibroblast cell adhesion experiments in terms of surface stretching and occupation superficial on: A) Surface after machining B) Surface after machining and anodising 100V; C) Surface after subtractive acid treatment D) Surface after subtractive acid treatment and anodising 100V.

FIG. 10 shows scanning electron microscopy images with retro-dispersed electrons representative of the occupation of the fibroblast cells on: A) Surface after machining B) Surface after machining and anodising 100V; C) Surface after subtractive acid treatment D) Surface after subtractive acid treatment and anodising 100V.

FIG. 11 shows the results of the gingival-based primary fibroblast cell differentiation experiments in terms of Type I procollagen and fibronectin synthesis [A) Surface after machining B) Surface after machining and anodising 100V C) Surface after subtractive acid treatment D) Surface after subtractive acid treatment and anodising 100V].

### DETAILED DESCRIPTION OF THE INVENTION

An object of the invention is a method for surface treatment of a dental implant or prosthetic component made out of titanium or a titanium alloy. This method comprises a step to provide an outer surface of the implant or prosthetic component with a surface roughness, and a subsequent step of applying an anodising treatment on the implant or prosthetic component, smoothing the roughness and generating nanopores on this outer surface of the implant or component. Nanopores are understood to be numerous holes with a diameter within a dispersion around an average diameter smaller than or equal to 300 nm, wherein the holes have a depth substantially equal or equivalent to the diameter and are distributed randomly covering the entire surface.

In some embodiments of the method according to the invention, the step of providing the implant or component with a surface roughness comprises creating a surface roughness through the machining of the implant or prosthetic component. In other embodiments, this surface roughness is created through a mechanical treatment of the implant or the component. In other embodiments, this surface roughness is created through a chemical treatment of the implant or the component, through a deposition process, or through a thermal treatment of the implant or the component. In other embodiments, the step of providing the implant or component with a surface roughness comprises creating a surface roughness through an electrochemical treatment of the implant or component.

In some embodiments of the invention, the step of applying an anodising treatment on the implant or component may comprise submerging the implant or component in an electrochemical bath of at least one electrolyte and subjecting this bath to a voltage. Electrolytes such as phosphoric acid (H3PO4), sulphuric acid (H2SO4), hydrofluoric acid (HF), oxalic acid (C2H204) or combinations of them may be used. For example, the electrochemical bath may contain between 1% and 50% of phosphoric acid (H3PO4). In another example, the electrochemical bath may contain between 1% and 3% of oxalic acid (C2H2O4). The voltage may be from 25 to 200 V, and preferably from 75 to 170 V, and even more preferably from 80 to 120 V. The voltage may preferably be applied for at least 1 second and less than 10 minutes.

In some embodiments, the step of applying an anodising treatment on the implant or component is carried out at a temperature whose value ranges from −25 to 100° C. For example, in certain embodiments, the step of applying an anodising treatment on the implant or component may be carried out at room temperature.

Another object of the invention is to provide a dental implant or prosthetic component, made out of titanium or a titanium alloy, which comprises a rough outer surface with nanopores. Nanopores are understood to be numerous holes with a diameter within dispersion around an average diameter smaller than or equal to 300 nm, wherein the holes have a depth substantially equal or equivalent to the diameter and are distributed randomly covering the entire surface. In some embodiments, the rough outer surface comprises a random distribution of circular pores with a diameter and depth varying between 10 and 300 nm.

The tests performed on the method according to the invention and the resulting products are described below.

### 1. Description of the Tests

### 1.1 Aesthetic and Topographic Evaluation of the Surfaces

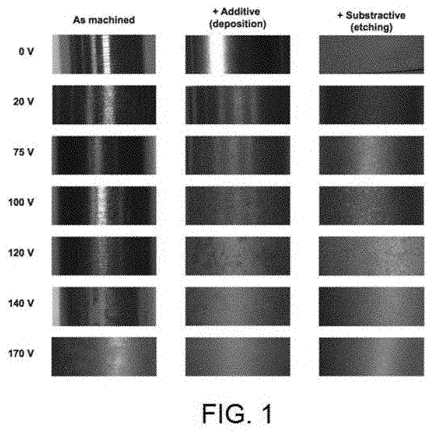

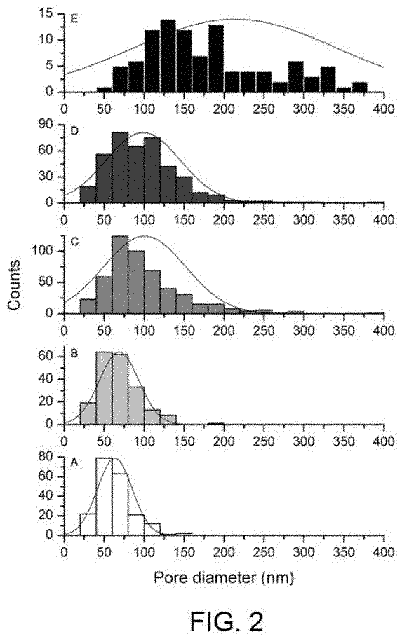

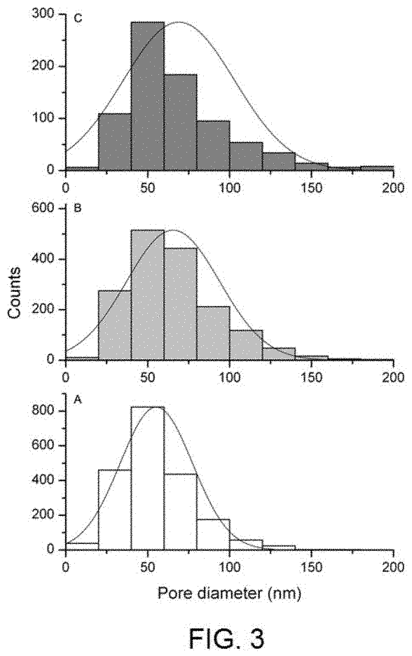

The surface nano-texture in this invention was generated on different pre-existing surfaces to evaluate the aesthetic and functional effect. For greater representativeness, three types of substrate were chosen: substrates without any modification after machining, or in other words, with the surface exactly how it is after using the lathing tool to form an implant; substrates of the same nature but to which an additive treatment has been applied in order to provide the surface with a harder finish (nitriding); and substrates of the same nature but to which a subtractive surface treatment has been applied (acid etching) in order to provide roughness in accordance with industry standards. To provide nano-texture, different anodising treatments were applied at different voltages on the three substrates. The aesthetic appearance of the different surfaces obtained was observed under optical microscopy, whereby the images obtained under the microscope are shown in FIG. 1. After consulting several prosthetic experts, the most favourable tones for the gingival area were those produced by the samples with nano-texture after the additive treatment (at 100V, 140V or 170V) or after the subtractive treatment (at 100V). In particular, the effect of the surface with additive treatment and subsequent nano-texturising at 100V can be highlighted as it generates pinkish reflections very similar to the natural tone of the gum. The pore diameter distribution histogram of the nano-textures on additive treatment (visible in FIG. 2) shows that as the anodising voltage is increased, the dispersion in the pore diameter also increases, whereby its average is around 60 nm (75V), 70 nm (100V), 100 nm (125V and 140V) and 210 nm (170V). The same occurs in the case of the nano-textures on subtractive treatment (FIG. 3) although the averages are slightly lower: 55 nm for 75V, 65 nm for 100V and 70 nm for 125 V.

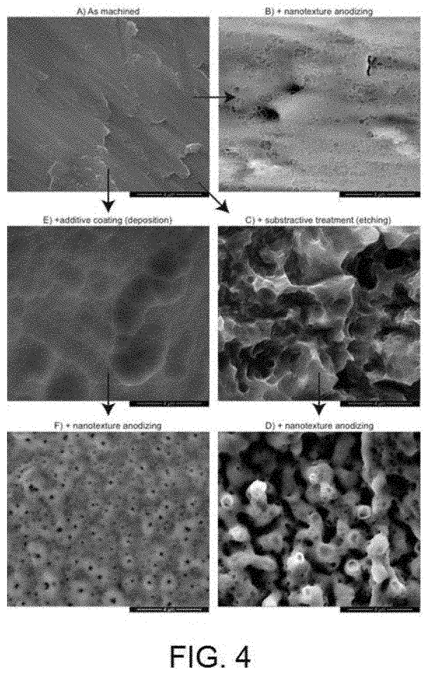

For the aesthetic results and greater homogeneity of the pore size at around 100 nm in diameter, surfaces with nano-texturised treatment through anodising at 100V were use for the following experiments. FIG. 4 shows the topographic appearance of these surfaces with respect to their predecessors (machining, subtractive or additive treatment) obtained using scanning electron microscopy at 20,000 augmentations. In all cases, it can be seen that after the nano-texturising treatment, the topographic characteristics of the pre-existing surface treatment can be seen to which the nanopores are added. The effect in the case of the additive treatment can be highlighted as the nano-texturising generates a surface with a more homogeneous and regular porosity distribution.

### 1.2 Quantification of the Bacterial Adhesion

The purpose of this series of tests is to compare the capacity of the surfaces with a porous nano-texture, object of this invention with the reference surfaces normally used in transepithelial components.

In an initial stage, in vitro experiments were performed, in static conditions, with two significant strains of general infectious processes (Staphylococcus Aureus) and more related to the oral cavity (Streptococcus Sanguinis). In all cases, the nano-texture enabled the adhesion of both bacterial strains to be significantly reduced in statistical terms in comparison with the controls without nano-texture.

Then, more complex and representative experiments were performed on the real functioning of the surfaces in the mouth through a dynamic bacterial adhesion model with artificial and natural saliva conditioning (obtained from healthy patients) and with strains representative of the oral cavity: the aforementioned Streptococcus Sanguinis, Streptococcus Mutans and Aggregatibacter Actinomycetemcomitans. In this case, only the machined surfaces and those with subtractive treatment with and without nano-texturising treatment were compared. It is worth mentioning that, unlike the static test, only the nano-texturised treatment on the previously modified surface with subtractive treatment (and not on the machined surface) obtained systematic results and significantly less bacterial adhesion regardless of the bacterial strain studied and the average was conditioned with artificial or natural saliva.

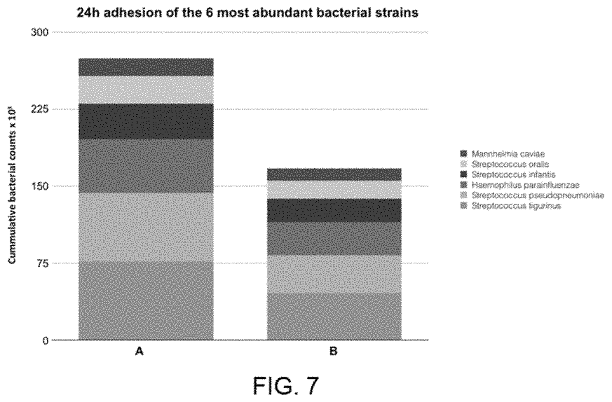

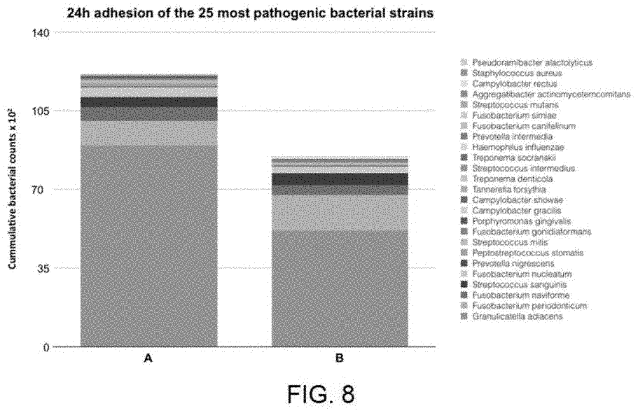

The next step was the in vivo evaluation of the adhesive capacity of the surfaces with and without nano-texture, in this case on surfaces with previously applied additive treatment. To do so, discs were laid out on modified and unmodified surfaces on ferrules specifically adapted to 6 patients and, after 24 h in the mouth, the amount of bacteria present was measured using metagenomics techniques. For the analysis of the data, the 6 most abundant bacteria in the mouth were initially selected (FIG. 7, whereby the results A and B correspond to the surfaces without and with nano-texture, respectively). Then the 25 most pathogenic bacteria related to infectious processes in the oral cavity (FIG. 8, whereby the results A and B correspond to the surfaces without and with nano-texture, respectively) were selected. The result, in both cases, led to a statistically significant reduction in bacterial adhesion in the presence of the nano-texture.

### 1.3 Evaluation of the Adhesion of Gingival Fibroblasts

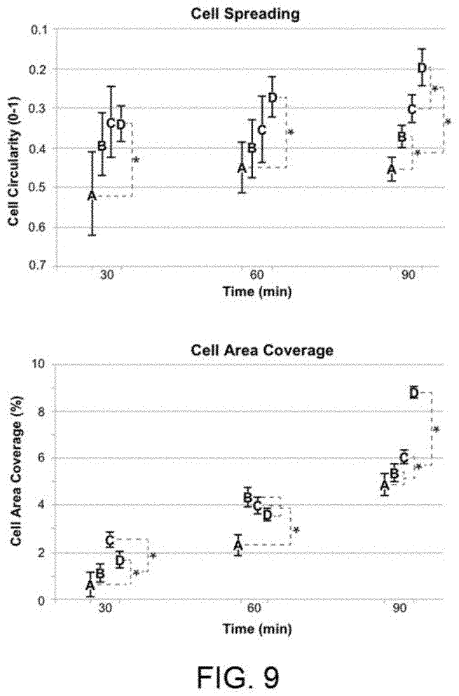

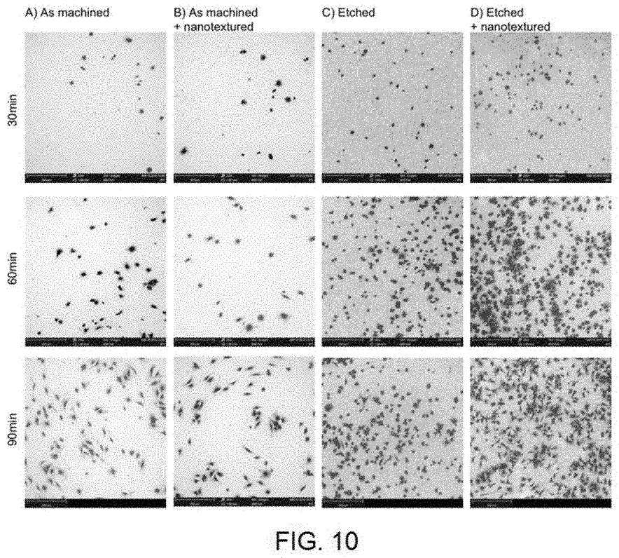

Once the increased rejection of bacterial adhesion of the surfaces with nano-texture had been determined, it is advisable to determine whether this rejection is not generalised to any cell, in particular to the cells of interest in the gingival area: the gingival fibroblasts. Therefore, adhesion and cell extension experiments were performed on solely machined discs and on discs with roughness as a result of subtractive treatment, whereby both types had surfaces with and without nano-texture. On one hand, the circularity of the cells is evaluated (as well as the reverse extension), which will show that the cells that have been adhered are well adhered and are functional and, on the other hand, the amount of the total area covered by the cells which shows the affinity of each type of surface due to the adhesion of this particular type of cells. The results which are shown in FIG. 9 (where A corresponds to machined surfaces without nano-texture, B corresponds to machined surfaces with nano-texture, C corresponds to surfaces with roughness by subtractive treatment and without nano-texture, and D corresponds to surfaces with roughness by subtractive treatment and with nano-texture) indicate that the pre-treated surfaces with nano-texture (D) enable a greater extension of the fibroblasts, especially when the cells are exposed to the surface for periods longer than 60 minutes. In the case of the surface coating, the pre-treated surfaces and with nano-texture (D) are those with a greater percentage of the surface occupied by the cells with a large differential at 90 minutes exposure, although in the previous times, the results are very similar among all of the surfaces with some type of surface treatment (B, C and D). The electron microscopy images in FIG. 10 support these results.

### 1.4 Evaluation of the Matrix Synthesis by the Gingival Fibroblasts

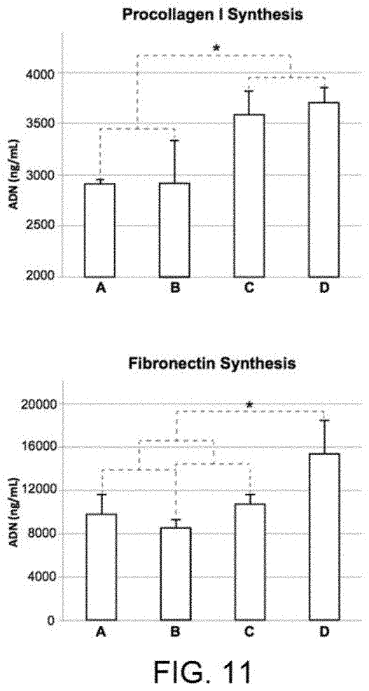

In addition to there being a greater number of cells and that these have a functional layout, or in other words, they are well stretched across the surface, specific tests measuring the protein released by the cells provide a quantification of the regenerating potential, or in other words, of the potential to create an extra-cellular matrix than that of the different surfaces studied. FIG. 11 (where A corresponds to machined surfaces without nano-texture, B corresponds to machined surfaces with nano-texture, C corresponds to surfaces with roughness by subtractive treatment and without nano-texture, and D corresponds to surfaces with roughness by subtractive treatment and with nano-texture) shows the cell differentiation results through the quantification of Type 1 procollagen and fibronectin synthesis. In the case of procollagen synthesis, no significant increase is observed when the surfaces are treated with nano-texture (B and D). Only the rougher surfaces with subtractive pre-treatment (C and D), regardless of the nano-texture, obtain statistically significant better results. As for fibronectin synthesis, only the treatment with nano-texture after the pre-treatment of the surface (D) obtains significantly greater results.

In conclusion, the adhesion and cellular differentiation with gingival fibroblast results show that the inhibition of adhesion is specific to bacteria and not to eukaryotes cells from the gingival tissue. Quite the opposite, the nano-texture added to the pre-treatment of the surface enables the inhibition of the bacterial adhesion of pathogenic elements from the oral cavity, increase the regenerating potential through a higher number of cells forming healthy tissue.

### 2. Experimental

### 2.1 Preparation of the Surfaces

For the experiments, discs with a diameter of 12.7 mm and thickness of 2 mm and others with a diameter of 6 mm and a thickness of 1 mm based on Grade 4 commercially pure titanium which is usually used in the manufacture of dental implants, were prepared. The machined surfaces correspond to the surface state following the machining (lathing) of the parts. Two types of surface treatment were performed on this control surface, which acted as a model. On one hand, a subtractive treatment was performed, consisting of the immersion of the machined pieces in an acid bath of concentrated H2SO4/HCl at 90° C. for 20 minutes and then in HNO3 at 15% and at room temperature for 20 minutes. On the other hand, an additive treatment was performed, consisting of the plasma vapour deposition (PVD) of a layer of 1 to 2 μm of titanium nitride. Nano-texturising was performed by submerging the discs in a bath of H3PO4 al 25% for 1 minute and applying a variable anodising voltage of between 20 and 170 V. These treatments were carried out on machined surfaces after subtractive treatment and after additive treatment. Following the preparation of each of the surfaces, the discs were immediately cleaned in Type A clean room conditions prior to their sterilisation in individual containers via irradiation by R rays for their storage prior to the tests.

### 2.2 Qualitative Evaluation of the Surfaces by Microscopy

Optical Microscopy: the qualitative observation of the aesthetic finish of the pieces was analysed under a Leica DMLB (Leica Microsystems, Wetzlar, Germany) optical microscope with an attached digital camera, Leica DFC300FX model and with a magnification of 10×.

Electron Microscopy: for the determination of the micro and the nanotopography, a scanning electron microscope was used (SEM, Quanta 200FEG, FEI Eindhoven, Netherlands) in secondary electron mode, with an acceleration voltage of 30 kV and a beam size of 5 Å at different magnifications between 1000× and 40000×.

### 2.3 Determination of the Pore Diameter

The evaluation of the average diameter of the pores was carried out based on scanning electron microscope images (See above) at 30000× augmentations in 10 different areas for each type of sample. The images were then processed using the ImageJ software with the application of a brightness/contrast filter that enabled the nanopores to be isolated from the rest of the image. A counting algorithm was them applied that enabled basic geometric aspects to be determined, such as the diameter of each pore. Once the data had been extracted, Origin software (v7.0654651) was used to calculate the pore diameter distribution histograms in accordance with the treatments applied.

### 2.4 In Vitro Microbiological Tests

Bacterial strains: The static tests were performed with the Staphylococcus aureus (S. aureus) ATCC29213 and Streptococcus sanguinis ATCC10556 (S. sanguinis) strains. The dynamic tests were performed with the Streptococcus mutans ATCC25175 (S. mutans), Streptococcus sanguinis ATCC10556 (S. sanguinis) and Aggregatibacter actinomycetemcomitans ATCC43718 (A. actinomycetemcomitans) strains.

Experimental Conditions: The bacteria was pre-cultivated on BHI agar plates without supplementation for 48 h, for S. Mutans, S. Sanguinis and S. aureus in an atmosphere of 5% CO2, and for 72 h for A. actinomycetemcomitans, in anaerobiosis conditions, at 37° C. It was then incubated for 24 h, for S. Mutans, S. Sanguinis and S. aureus in 100 ml of BHI, or for 48 h for A. actinomycetemcomitans in 200 ml of BHI, at 37° C. The indicated average bacterial growth times and volumes correspond to the optimum viability and growth conditions to carry out the experiments and they were selected after analysing several different times. The concentration of bacteria in the suspensions was 10⁸ bacteria/ml, determined with a Neubauer camera. To come into contact with the substrates, the bacteria was suspended in artificial saliva (Jean-Yves Gal, 2001) free of proteins and with a pH value of 6.8. The static adhesion was performed at 37° C. for 60 min. The experimental device used for the dynamic adhesion was a Robbins camera with 9 ports which allowed 9 samples to be analysed simultaneously and in laminar flow conditions, at a speed of 2 ml/min and at physiological temperature. Prior to the initial adhesion tests, a study was carried out to determine which of the positions of the Robbins device would not influence the final adhesion results. The dynamic adhesion experiment was performed uninterruptedly for 60 min, and once completed, the adhesion and viability was quantified. In this case, all of the experiments were carried out simultaneously for all of the substrates (machined surfaces with and without nano-texture, and surfaces with prior roughness due to subtractive treatment with and without nano-texture). The final analysis final of the adhesion was performed using fluorescence microscopy with a LIVE/DEAD BacLight™ viability kit. The dynamic experiments were grouped into two groups: a first group, considering the direct response of the material, in which case the samples were placed directly in the Robbins device without prior conditioning; a second group, considering the response of the material with prior conditioning, in which case the samples were subjected to 60 min conditioning with natural saliva (Sánchez MC, 2011), from a pool obtained from young and healthy volunteers of both sexes, including smokers and non-smokers. All of the experiments were carried out three times and with independent crops. For each substrate, the viability and adhesion in 6 different positions on the surface has been studied.

Statistical Analysis: The statistical study has been performed using analysis of variance (ANOVA) and Student's T-test to verify whether to accept the null hypothesis that the averages of different populations coincide. On carrying out the ANOVA or Student's T-test for independent samples, if a low signification is obtained (less than 0.05), the hypothesis that the averages of the groups are the same is rejected. In the analysis of variance (ANOVA), to identify in which groups the differences have occurred, the unplanned contrasts or post-hoc contrasts have been used, which are used when there is no prior idea of which groups to expect the biggest differences. This analysis is considered to be quite conservative, given that the differences between groups must be really large to be detected, so it is likely that there are situations in which there are subtle differences between groups that are not detected by the post-hoc tests. Multiple comparison techniques have been used, which seek to establish differences between groups based on paired differences. In this analysis “Tukey's Honestly Significant Difference” (HSD Tukey) test and the Games-Howell test have been used, which are techniques that allow each group to be compared with all the rest when the number of groups is high. The size of each group is the number of images that have been captured and analysed under the fluorescence microscope for each surface treatment: 6 regions per test specimen and as all of the experiments are performed three times, 18 figures/group. All of the groups are of the same size. For all of the calculations, the SPSS v12 (Chicago, Ill., USA) statistical programme has been used.

### 2.5 In Vivo Microbiological Tests

The discs with the different study surfaces were placed in polycarbonate ferrules specifically designed to hold them and adapted to the upper maxillary of 6 healthy patients between the ages of 24 and 45. The work surfaces were facing the mouth area, above the teeth. Two discs were positioned on each side, alternating the positions in accordance with the two surfaces being tested: without and with nano-texturising treatment. The ferrules were worn continuously in the mouth for 24 h and were only removed to eat and clean teeth. Afterwards, the discs were removed from the ferrules, rinsed with plenty of water to eliminate any traces that were not adhered and they were stored at −80° C. until their analysis.

### Metagenomic Analysis and Sequencing of the 16S Ribosome

Metagenomic studies are usually performed through the analysis of the 16S ribosomal RNA (16S rRNA) gene, which contains around 1500 base pairs (bp) and contains nine variable regions interspersed with conserved regions. The variable regions of the 16S rRNA gene are often used for phylogenetic classifications, such as gender or species in diverse microbial populations. This metagenomic analysis protocol is based on the sequencing and analysis of the variable regions V3 and V4 of the 16S rRNA gene. This protocol combines the MiSeq (Illumina) sequencing system, with primary and secondary analyses using specific IT packages and biocomputing tools in order to generate a complete metagenomic analysis strategy of the 16S rRNA, The protocol includes five different phases:

1. Isolation of the microbial DNA. The microbial DNA was obtained from the surface of the discs subjected to the test using a specific DNA isolation kit which enables the DNA to be isolated from all types of biofilm samples with a high level of quality. Then, the DNA samples were quantified using spectrophotometry and fluorimetric analysis.

2.—Polymerase chain reaction (PCR) amplification of the target sequences. The sequences for the first pair in the V3 and V4 region create a unique amplicon of around ˜460 bp. Along with these primers, sequences of specific adapters are added for compatibility with the Illumina index and the sequencing adapters.

3.—Preparation of the library. Once the selected V3 and V4 region has been amplified, the Illumina sequence adapters and the dual index bar codes are added and to the target amplicon. This protocol allows up to 96 libraries to be joined together in the same sequence.

4.—MiSeq Sequencing. Using the MiSeq reagents and the base pair readings 300-bp, the full reading of the V3 and V4 region is sequenced. MiSeq produces approximately >20 million readings and can generate >100,000 readings per sample, taking into consideration 96 indexed samples.

5.—Biocomputing Analysis. Once the sequences have been generated, a secondary analysis is performed following the metagenomic flow for taxonomic classification, using the available databases. This allows for the bacterial classification in accordance with the gender or species.

### 2.6 Tests with Fibroblastic Cells

Primary cells of human gingival fibroblasts were cultivated as described in Anitua E, Tejero R, Zalduendo M M, Orive G. Plasma rich in growth factors promotes bone tissue regeneration by stimulating proliferation, migration, and autocrin secretion in primary human osteoblasts. J Periodontal 2013:84:1180-90. Briefly, the gingival fibroblasts are stored in Eagle crop modifying Dulbecco (DMEM)/F12 (Gibco-lnvitrogen, Grand Island, N.Y., US) and supplemented with glutamine 2 mM, gentamicin 50 μg ml⁻¹ (Sigma) and 15% of fetal bovine serum. (FBS) (Biochrom AG, Leonorenstr, Berlin, Germany). The crops were incubated in a humidified atmosphere at 37° C. and 5% CO2. For the experiments, the cells between the fourth and sixth step were selected. Three replicas were used for each type of surface and experiment.

### Cellular Adhesion and Extension

The cells were planted in the culture medium with a density of 20000 cells cm⁻² for 30, 60 and 90 min. On completion of these times, the culture medium was discarded and the wells were rinsed with phosphate bufferedsaline (PBS) serum. The level of cell coating on the surfaces was measured via electron microscopy images taken with an electron acceleration voltage of 5 kV. The samples were previously fixed for 12-15 hours in glutaraldehyde at 3 wt. % and were later washed 3×10 min with PBS (pH=7.4). Then, the samples were dehydrated through the application of ethanol increasing concentration solutions (30, 50, 70, 90 and 100 vol. %). The samples were in each concentration for 60 min. To analyse the percentage of the area covered by the cells in the different surfaces, ImageJ software was used. The cell extension was calculated as the inverse of their circularity level.

### Release of Proteins From the Extra-Cellular Matrix

The discs with the different surfaces were placed on polystyrene cellular cultivation plates. The cells were cultivated on them with the full half and with a density of 6000 cells cm⁻². After 7 days of cultivation, ELISA kits (Takara, Shiga, Japan) were used to determine the fibronectin and the Type 1 procollagen synthesis.

1. Method for surface treatment of a dental implant or a prosthetic component made out of titanium or a titanium alloy, characterised in that it comprises the following steps:
providing an outer surface of the implant or the component with a surface roughness; and
applying an anodising treatment on the implant or the component, smoothing the roughness and generating nanopores with a diameter and depth smaller than or equal to 300 nm on this outer surface of the implant or the component.

2. Method, according to claim 1, characterised in that the step of providing the implant or the component with a surface roughness comprises creating a surface roughness through the machining of the implant or the component.

3. Method, according to claim 1, characterised in that the step of providing the implant or the component with a surface roughness comprises creating a surface roughness through a mechanical treatment of the implant or the component.

4. Method, according to claim 1, characterised in that the step of providing the implant or the component with a surface roughness comprises creating a surface roughness through a chemical treatment of the implant or the component.

5. Method, according to claim 1, characterised in that the step of providing the implant or the component with a surface roughness comprises creating a surface roughness through a deposition process.

6. Method, according to claim 1, characterised in that the step of providing the implant or the component with a surface roughness comprises creating a surface roughness through a thermal treatment of the implant or the component.

7. Method, according to claim 1, characterised in that the step of providing the implant or the component with a surface roughness comprises creating a surface roughness through an electrochemical treatment of the implant or the component.

8. Method, according to claim 1, characterised in that the step of applying an anodising treatment on the implant or the component comprises submerging the implant or the component in an electrochemical bath of at least one electrolyte and subjecting this bath to a voltage.

9. Method, according to claim 8, characterised in that at least one electrolyte comprises hydrofluoric acid (HF).

10. Method, according to claim 8, characterised in that at least one electrolyte comprises sulphuric acid (H2SO4).

11. Method, according to claim 8, characterised in that at least one electrolyte comprises phosphoric acid (H3PO4).

12. Method, according to claim 11, characterised in that the electrochemical bath comprises between 1% and 50% of phosphoric acid (H3PO4).

13. Method, according to claim 8, characterised in that the at least one electrolyte comprises oxalic acid (C2H204).

14. Method, according to claim 13, characterised in that the electrolyte comprises between 1 and 3% of oxalic acid (C2H2O4).

15. Method, according to claim 8, characterised in that the voltage presents a value from 25 to 200 V.

16. Method, according to claim 15, characterised in that the voltage presents a value from 75 to 170 V.

17. Method, according to claim 16, characterised in that the voltage presents a value from 80 to 120 V.

18. Method, according to claim 8, characterised in that the voltage is applied for at least 1 second.

19. Method, according to claim 8, characterised in that the voltage is applied for less than 10 minutes.

20. Method, according to claim 8, characterised in that the step of applying an anodising treatment on the implant or the component is carried out at a temperature whose value is from −25 to 100° C.

21. Method, according to claim 8, characterised in that the step of applying an anodising treatment on the implant or the component is carried out at room temperature.

22. Dental implant or prosthetic component, made out of titanium or a titanium alloy, characterised in that it comprises a rough outer surface with nanopores of a diameter and depth smaller than or equal to 300 nm.

23. Dental implant or prosthetic component, according to claim 22, characterised in that this rough outer surface comprises a random distribution of circular pores with a diameter and depth of between 10 and 300 nm.
